(19) United States
(12) Patent Application Publication
(10) Pub. No.: US 2022/0387709 A1
(43) Pub. Date: Dec. 8, 2022

(54) SYSTEMS AND METHODS FOR COMPENSATION OF TUBING STRESS RELAXATION EFFECTS WITH INFUSION PUMP SYSTEMS

(71) Applicant: Smiths Medical ASD, Inc., Plymouth, MN (US)

(72) Inventors: Quinn Morrison, Minneapolis, MN (US); Paul Harrison Coons, Sunfish Lake, MN (US)

(21) Appl. No.: 17/774,243

(22) PCT Filed: Nov. 2, 2020
(86) PCT No.: PCT/US2020/070734

§ 371 Date: May 4, 2022

Related U.S. Application Data
(60) Provisional application No. 62/930,341, filed on Nov. 4, 2019.

(51) Int. Cl. A61M 5/168; A61M 5/142

(52) U.S. Cl. CPC A61M 5/16854; A61M 5/14228; A61M 2005/16863; A61M 2205/3331

(57) ABSTRACT

An infusion pump including an administration set configured to provide a fluidic pathway between a supply of infusate and an infusion set, at least one pressure sensor configured to sense a pressure of infusate within the administration set, and a control unit configured to monitor the sensed pressure, and apply a calculated tare adjustment to the monitored pressure to compensate for a decay of observable stress within the administration set as a result of stress relaxation.

### RELATED APPLICATION

The present application claims the benefit of U.S. Provisional Application No. 62/930,341 filed Nov. 4, 2019, which is hereby incorporated herein in its entirety by reference.

### TECHNICAL FIELD

The present disclosure relates generally to infusion pup systems, and more particularly, to systems and methods for compensation of tubing stress relaxation effects with infusion pump systems.

### BACKGROUND

Various types of infusion pumps have been useful for managing the delivery and dispensation of a prescribed amount or dose of a drug, fluid, fluid-like substance, or medicament (herein, collectively, an “infusate”) to patients. Infusion pumps provide significant advantages over manual administration by accurately delivering infusates over an extended period of time. Infusion pumps are particularly useful for treating diseases and disorders that require regular pharmacological intervention, including cancer, diabetes, and vascular, neurological, and metabolic disorders. Infusion pumps also enhance the ability of healthcare providers to deliver anesthesia and manage pain. Infusion pumps are used in various settings, including hospitals, nursing homes, and other short-term and long-term medical facilities, as well as in residential care settings. Types of infusion pumps include ambulatory, large-volume, patient controlled anesthesia (PCA), elastomeric, syringe, enteral, and insulin pumps. Infusion pumps can be used to administer medication through various delivery methods, including intravenously, intraperitoneally, intra-arterially, intradermally, subcutaneously, in close proximity to nerves, and into an inter-operative site, epidural space, or subarachnoid space.

In a particular type of infusion pump system that is commonly referred to as a “peristaltic” pump system, delivery of an infusate to a patient is typically accomplished with the use of an infusion administration set, that is typically disposed of after use and can provide a fluidic pathway (e.g., tubing) for the infusate from a reservoir (such as an intravenous or “IV” bag) to a patient, in cooperation with a pump that controls the rate of flow of the infusate. Peristaltic infusion pumps incorporate a peristaltic pumping mechanism that can function by repetitively and temporarily occluding successive sections of tubing of the administration set in a wave-like motion. A “large-volume pump” or “LVP” system is a common type of peristaltic pump with related components as aforedescribed; in some publications, the term “volumetric pump” may also be variously used to refer to an LVP or peristaltic pump.

Frequently, LVPs include one or more sensors configured to monitor a fluid pressure of the infusate within the tubing. For example, LVPs often include a “downstream pressure sensor” configured to detect an unwanted occlusion to a prescribed flow of infusate outwardly from the LVP, and an “upstream pressure sensor” configured to detect when the reservoir is empty and/or when an otherwise abnormal fluid pressure is present upstream of the pumping mechanism. These sensors typically must provide relatively high reliability and high sensitivity to ensure that the desired amount of infusate is being delivered to the patient.

The accurate detection of pressure within the tubing can be complicated by characteristics of the tubing itself. In particular, certain materials used in the construction of intravenous infusion tubing can be subject to a phenomenon commonly referred to as “stress relaxation.” Stress relaxation generally refers to a decay of observable stress in the tubing wall as the tubing is held under pressure, thereby making it difficult to accurately monitor fluid pressure data over time. Frequently, the tubing returns to its original shape upon removal of the pressure load, such as from a peristaltic pumping mechanism acting upon it. Over time the stress relaxes due to changes in the material properties of the tubing, which results in a false appearance of a decrease in the infusate pressure as measured by the pressure sensor.

Maintaining catheter patency and minimizing occlusions is important for enhancing patient safety and improving therapeutic outcomes. In order to alert users to the presence of an occlusion, many infusion pump systems such as LVPs have a preset occlusion pressure threshold. Once the pressure sensed by the downstream pressure sensor exceeds the preset limit, an occlusion alarm is triggered. Although modern infusion systems are often quite adept at detecting occlusions, incorrect pressure detection as a result of stress relaxation can lead to an inability of a particular infusion system to detect occlusions at lower pressures. Higher occlusion pressures increase the potential for catastrophic tissue or organ injury in patients. Accordingly, relatively precise pressure detection can be crucial for the safe performance of infusion pumps.

The present disclosure addresses these concerns.

### SUMMARY OF THE DISCLOSURE

Embodiments of the present disclosure provide systems and methods for compensation of tubing stress relaxation effects, in sensing and measurement of a fluid pressure through a polymeric tube of an administration set, thereby enabling safer and more reliable sensing and measurement of infusate fluid pressures and an overall decrease in the amount of time necessary to detect an occlusion.

An embodiment of the present disclosure provides an infusion pump including an administration set configured to provide a fluidic pathway between a supply of infusate and an infusion set, at least one pressure sensor configured to sense a pressure of infusate within the administration set, and a control unit configured to monitor the sensed pressure, and apply a calculated tare adjustment to the monitored pressure to compensate for a decay of observable stress within the administration set as a result of stress relaxation.

In an embodiment, the infusion pump further includes a pump drive mechanism configured to urge infusate through the administration set by temporarily compressing a segment of the administration set. In an embodiment, the pump drive mechanism comprises a peristaltic drive mechanism. In an embodiment, the at least one pressure sensor comprises an upstream pressure sensor positioned upstream of the pump drive mechanism, a downstream pressure sensor positioned downstream of the pump drive mechanism a combination of an upstream pressure sensor and a downstream pressure sensor. In an embodiment, the control unit monitors the sensed pressure, in part, to detect the presence of an occlusion.

In an embodiment, the tare adjustment is calculated by the control unit based at least in part on data gathered by the at least one pressure sensor. In an embodiment, the tare adjustment is represented by a nonlinear function. In an embodiment, the tare adjustment is represented by a polynomial equation. In an embodiment, the tare adjustment is represented by the equation: R(t)=C₀R(t)=C₀+C₁·t^−τ₁+C₂^(−t/τ₂)+C₃^(−t/τ₃), wherein R equals the stress relaxation as a function of time (t), τ₁, τ₂, τ₃, C₂ and C₃ represent hardware characterization constants, and C₀, and C₁ represent fitted constants. In an embodiment, the tare adjustment is compared to acceptance criteria prior to application by the control unit.

An embodiment of the present disclosure provides an infusion pump including an infusion pump comprising a drive mechanism, at least one pressure sensor and a control unit, and an administration set configured to provide a fluidic pathway between a supply of infusate and an infusion set, wherein the infusion pump is configured to monitor a pressure within the administration set as sensed by the at least one pressure sensor, and apply a calculated tare adjustment to the monitored pressure to compensate for a decay of observable stress within the administration set as a result of stress relaxation.

Another embodiment of the present disclosure provides a method, including monitoring at least one of an upstream pressure and downstream pressure of an administration set via one or more sensors; calculating a tare adjustment based at least in part on data gathered from the one or more sensors; determining whether the calculated tare adjustment meets acceptance criteria; and applying the calculated tare adjustment to data gathered from the one or more sensors to compensate for a decay of observable stress within the administration set as a result of stress relaxation.

The summary above is not intended to describe each illustrated embodiment or every implementation of the present disclosure. The figures and the detailed description that follow more particularly exemplify these embodiments.

### BRIEF DESCRIPTION OF THE DRAWINGS

The disclosure can be more completely understood in consideration of the following detailed description of various embodiments of the disclosure, in connection with the accompanying drawings, in which:

FIG. 1 is a schematic perspective view depicting a peristaltic infusion pump system for use with a patient, in accordance with an embodiment of the disclosure.

FIG. 2A is a schematic perspective view depicting portions of a peristaltic infusion pump in the system of FIG. 1, particularly illustrating an assembly receptacle and receptacle door of the pump, in accordance with an embodiment of the disclosure.

FIG. 2B is a schematic perspective view depicting portions of the peristaltic infusion pump of FIG. 2A, with a portion of an administration set received by the assembly receptacle, in accordance with an embodiment of the disclosure.

FIG. 3 is a schematic view depicting various components and electrical circuitry of the peristaltic infusion pump in the system of FIG. 1, in accordance with an embodiment of the disclosure.

FIG. 4 is a cross-sectional view depicting stress distribution within a tube wall of an administration set, in accordance with an embodiment of the disclosure.

FIG. 5 is a graphical representation depicting a nonlinear degradation of stress at a given point in a tube wall of an administration set over a period of time as a result of stress relaxation, in accordance with an embodiment of the disclosure.

FIG. 6 is a graphical representation depicting a reaction force as measured by a sensor responsive to an exterior of the tube wall of an administration set over a period of time as a result of stress relaxation, in accordance with an embodiment of the disclosure.

FIG. 7 is a graphical representation depicting a calculation or curve fitting of a stress relaxation function using a method of least squares.

FIG. 8 is a graphical representation depicting a comparison between a compensated pressure sensor value to account for stress relaxation, an uncompensated pressure sensor value, and data from an external source configured to directly monitor an infusate pressure, in accordance with an embodiment of the disclosure.

FIG. 9 is a flowchart depicting a method for compensating for stress relaxation in the measurement of infusion pressures, in accordance with an embodiment of the disclosure.

While embodiments of the disclosure are amenable to various modifications and alternative forms, specifics thereof shown by way of example in the drawings will be described in detail. It should be understood, however, that the intention is not to limit the disclosure to the particular embodiments described. On the contrary, the intention is to cover all modifications, equivalents, and alternatives falling within the spirit and scope of the subject matter as defined by the claims.

### DETAILED DESCRIPTION

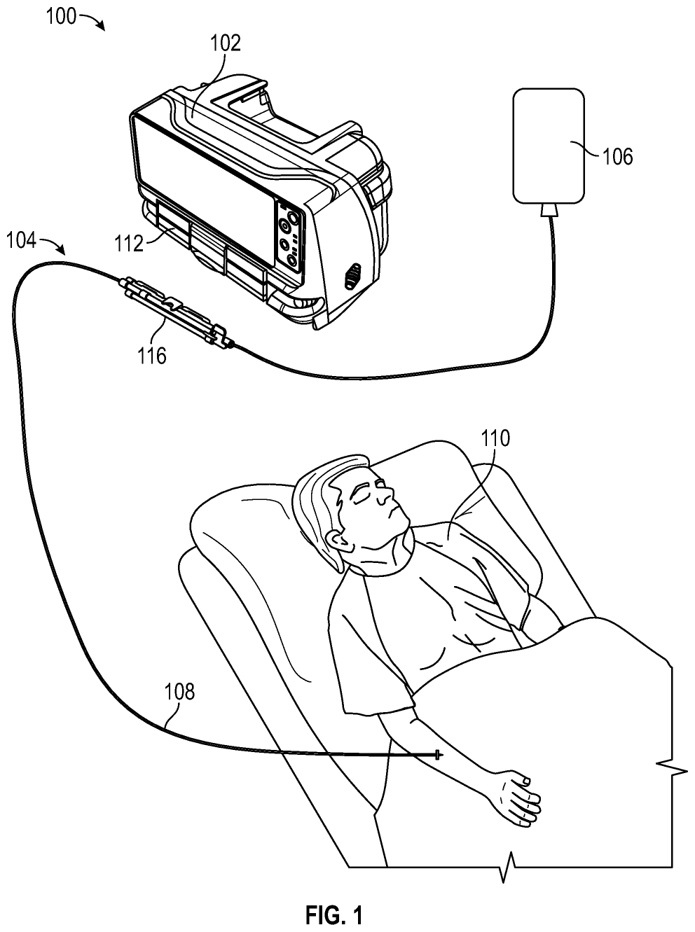

FIG. 1 is a schematic perspective view of an example embodiment of an infusion pump system 100 for use with a patient, that includes a peristaltic pump 102 (more specifically, a large volume pump or LVP 102) and an administration set 104 that may be disposable and structured and configured to be operably and removably coupleable to the pump 102. Administration set 104 is schematically shown providing a fluidic pathway from an IV bag 106 to an infusion set or tubing 108 that ultimately delivers infusate(s) to a patient 110. In FIG. 1, a receptacle door 112 of the pump 102 is shown in a closed configuration and the administration set 104 is illustrated as not coupled to the pump 102.

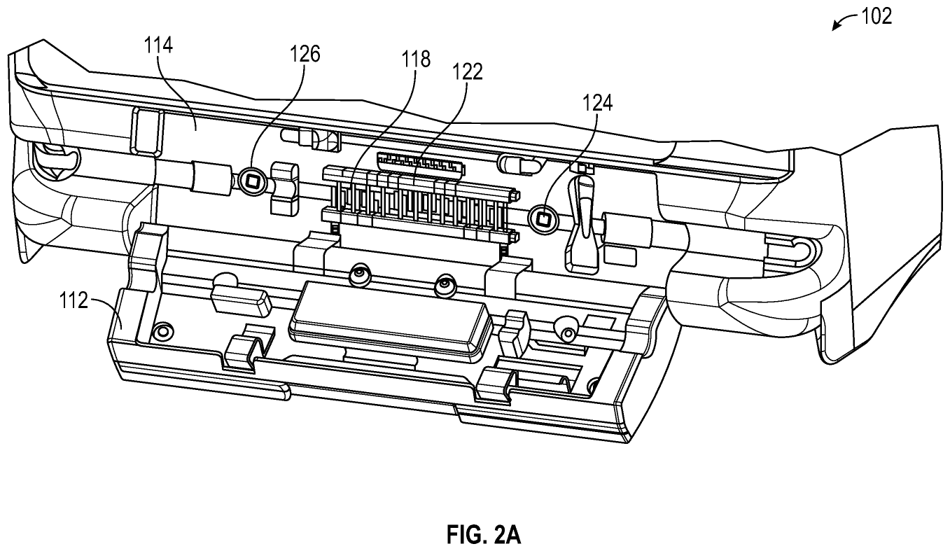

To more fully illustrate various components of the pump 102, FIG. 2A and FIG. 2B show a partial depiction of the pump 102. Specifically, only a portion of the pump 102 in proximity to an assembly receptacle 114 and receptacle door 112 is shown. The assembly receptacle 114 can be configured to receive an assembly 116 of the administration set 104, such that the administration set 104 is thereby operably coupled to the pump 102. In particular, FIG. 2B is a schematic perspective view of portions of the pump 102 of FIG. 2A, with assembly 116 received by or removably installed in the assembly receptacle 114. The receptacle door 112 can be opened or closed to allow or block access to the assembly receptacle 114. In both FIGS. 2A-B, the receptacle door 112 of the pump 102 is shown in an open position.

A peristaltic pump drive mechanism 122 can be located in the assembly receptacle 114. Assembly 116 of the administration set 104 can be configured and structured to position elements of the administration set 104, including a centrally located segment of a tube 120 of the assembly 116 in an operative relationship with the peristaltic drive mechanism 122. The centrally located segment of the tube 120 can be formed of a resilient material that is suitable for compression (and recovery from compression) by the peristaltic drive mechanism 122 of the pump 102. The peristaltic drive mechanism 122 can include tube engaging members 118 (sometimes referred to as “fingers”) that are configured to urge, push, force, or otherwise act to transport fluid through the administration set 104 by repetitively and temporarily squeezing or occluding the centrally located segment of tube 120 in a wave-like motion.

FIGS. 2A-B depict the pump 102 including twelve tube engaging members 118; in other embodiments, fewer or additional tube engaging members may be present. In general, the number of tube engaging members 118 determines the quantity of fluid delivery for each pump cycle or the “packet size” of fluid being delivered. For example, in an embodiment, the packet size of fluid can be about 150 μL; other packet sizes are also contemplated.

Fluid pressure generated within the administration set 104 is generally detectable via an elastic stretching or deformation of portions of the administration set 104. For example, in an embodiment, fluid pressures within the administration set 104, upstream and downstream of the tube engaging members 118, is detectable by an upstream pressure sensor 124 and a downstream pressure sensor 126, respectively. As depicted in FIGS. 2A-B, the upstream pressure sensor 124 and downstream pressure sensor 126 can be located within the assembly receptacle 114 on each respective side of the tube engaging members 118. Other locations, combinations and arrangements of sensors are also contemplated.

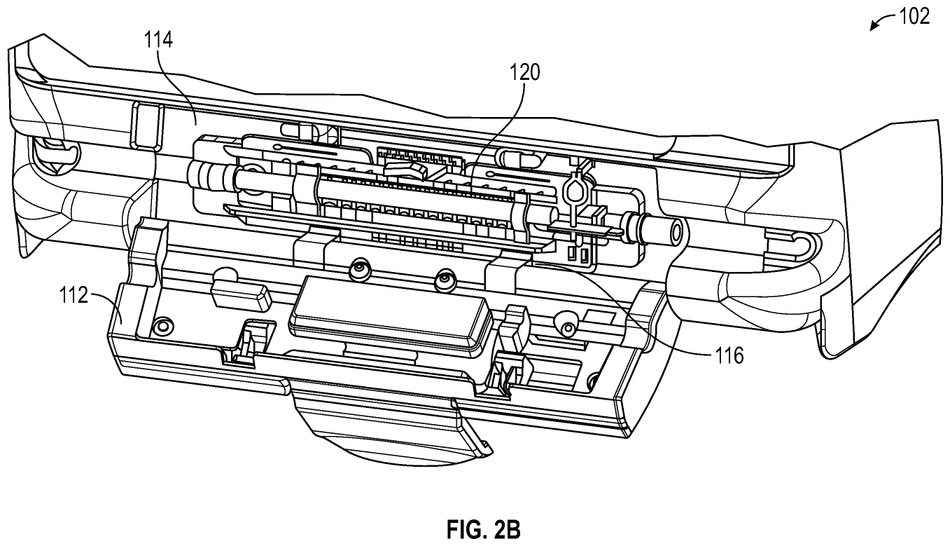

FIG. 3 is a schematic view of various components and electrical circuitry of the infusion pump 102 in the system 100. The tube engaging members 118 can be driven by the peristaltic drive mechanism 122, which can be controlled by a control unit 128 having a memory 129. The control unit 128 can receive inputs from a keypad 130, and other input devices, sensors and monitors, such as the upstream pressure sensor 124 and the downstream pressure sensor 126. The control unit 128 can also provide an output and receive input from a graphical user interface 132 such as, for example, a touch-screen input and display system.

In an embodiment, the control unit 128 can continually sense an upstream and a downstream pressure via the respective upstream pressure sensor 124 and downstream pressure sensor 126 to monitor for an occlusion and other infusate pressures which may indicate other than normal operation. Accurate detection of the infusate pressure within the tubing 108 can be complicated by characteristics of the tubing 108 itself. In particular, the centrally located tubing segment 120 of the assembly 116, which can be constructed of a suitable compressible resilient material, such as silicone, polyvinyl chloride, polyurethane, latex or rubber can be subject to a phenomenon commonly referred to as stress relaxation. Stress relaxation generally refers to a nonlinear decay of observable stress in the tubing segment 120 as the tubing segment 120 is held under pressure, thereby making it difficult to accurately monitor infusate pressure data over time as aforementioned.

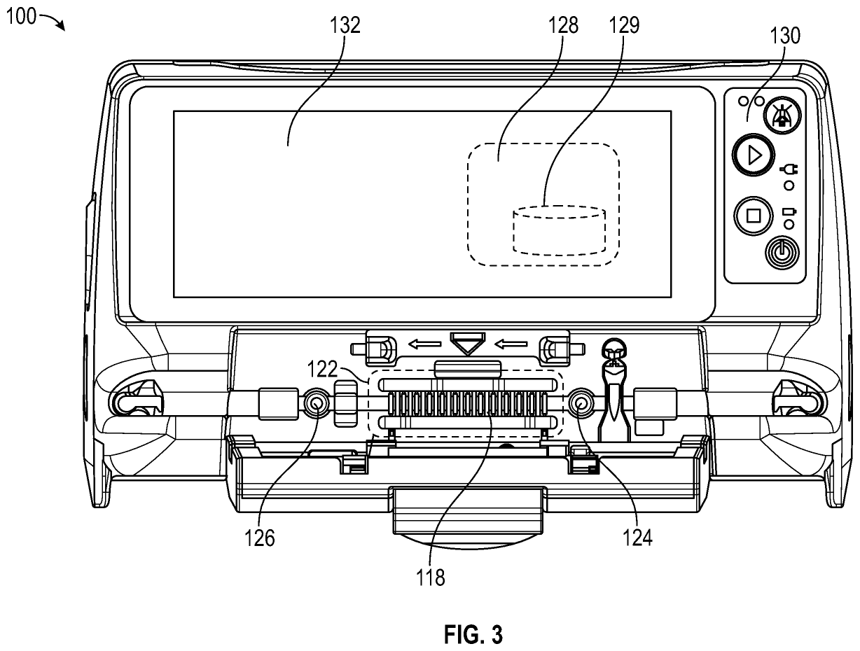

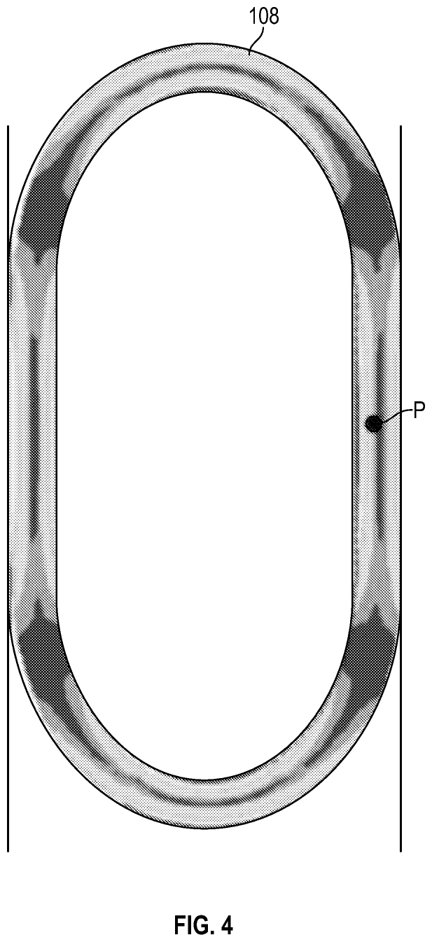

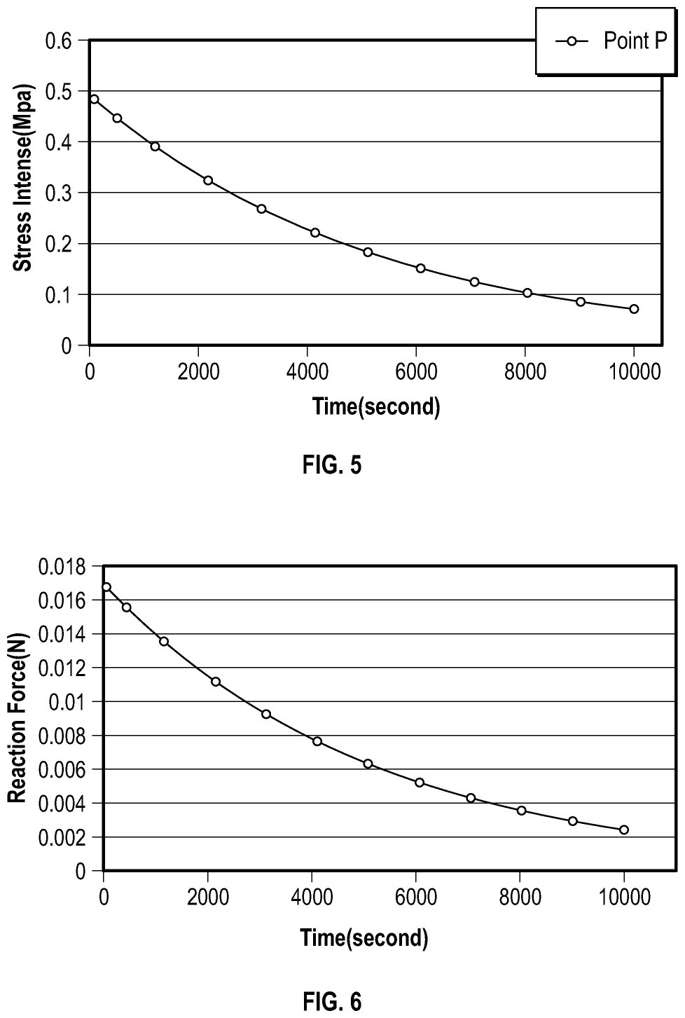

FIG. 4 depicts an initial stress distribution within a cross-section of tubing 108 after initial fluid pressurization. Accordingly, a finite stress is present within the cross-section of the tubing 108 at point P. FIG. 5 depicts a nonlinear degradation of the stress at point P over a period of time, as a result of stress relaxation in the tubing 108. FIG. 6 depicts a corresponding reaction force as measured by either the upstream pressure sensor 124 or the downstream pressure sensor 126 over the same period of time. As depicted in FIGS. 5 and 6, it is to be appreciated and understood that over time the stress in the tubing 108 relaxes which results in the false appearance of a decrease in the infusate pressure as measured by the pressure sensors 124 and/or 126. In some cases, the stress and corresponding reaction force can decrease by, for example, more than 20% in comparison to an initial stress and corresponding reaction force. These differences can present a significant error in monitoring infusate pressures during operation of pump 102. To further complicate matters, material of the tubing 108 typically returns to its original shape upon removal of the pressure load.

Embodiments of the present disclosure compensate for tubing stress relaxation effects in the administration set 104 by a continual, nonlinear adjustment of a so-called “tare” value, which represents a force magnitude as measured by the pressure sensors 124 and/or 126 associated with infusate in an unpressurized state (i.e., when the infusate is under ambient pressure conditions and not being subjected to effects of, e.g., operation of tube engaging members 118). In some embodiments, during operation, the tare can be adjusted over time based on a function to compensate for stress relaxation effects. In some embodiments, the tare can be reset to its initial value upon removal of the pressure load on the tubing material 108 (e.g., at the completion of an infusion pump cycle in operation of tube engaging members 118), to account for the material of the tubing 108 returning to its original shape.

In an embodiment, compensation for the stress relaxation effect can be provided according to the following polynomial equation:

R(t)=C₀+C₁t^{-τ}^{₁}+C₂^{(t/τ}^{₂}⁾+C₃^{(t/τ}^{₃}⁾

where, R equals the stress relaxation as a function of time (t), τ₁, τ₂, τ₃, C₂ and C₃ represent hardware characterization constants, and C₀, C₁ represent fitted constants as determined by the control unit 128 to approximate the stress relaxation function for a measurable stress relaxation under a constant infusate pressure over an initial trial period of time.

For example, in an embodiment, the values of τ₁, τ₂, τ₃, C₂ and C₃ are predetermined constants developed through hardware characterization prior to operation. Data gathered by the upstream sensor 124 and/or the downstream sensor 126 over an initial trial period of time (e.g., 500 seconds) can be used by the control unit 128 to calculate the values of C₀, and C₁ before an infusion operation is started in system 100, in order to fit the stress relaxation function (i.e., tare adjustment) to data observed by the sensors 124 and/or 126. Thereafter, the control unit 128 can adjust the tare, periodically or continually, according to the stress relaxation function over time.

It is to therefore be appreciated and understood that the tare function improves the estimate of the infusate pressure by accounting for the stress relaxation which would have otherwise created error in the measurement. By calculating values of C₀, and C₁ before each infusion, the tare function also can account for additional sources of variation, e.g. dimensional variation in the pump which may cause the initial reaction force, i.e. before stress relaxation, to be higher or lower. This can further improve the estimate of the infusate pressure and can improve the ability to detect occlusions.

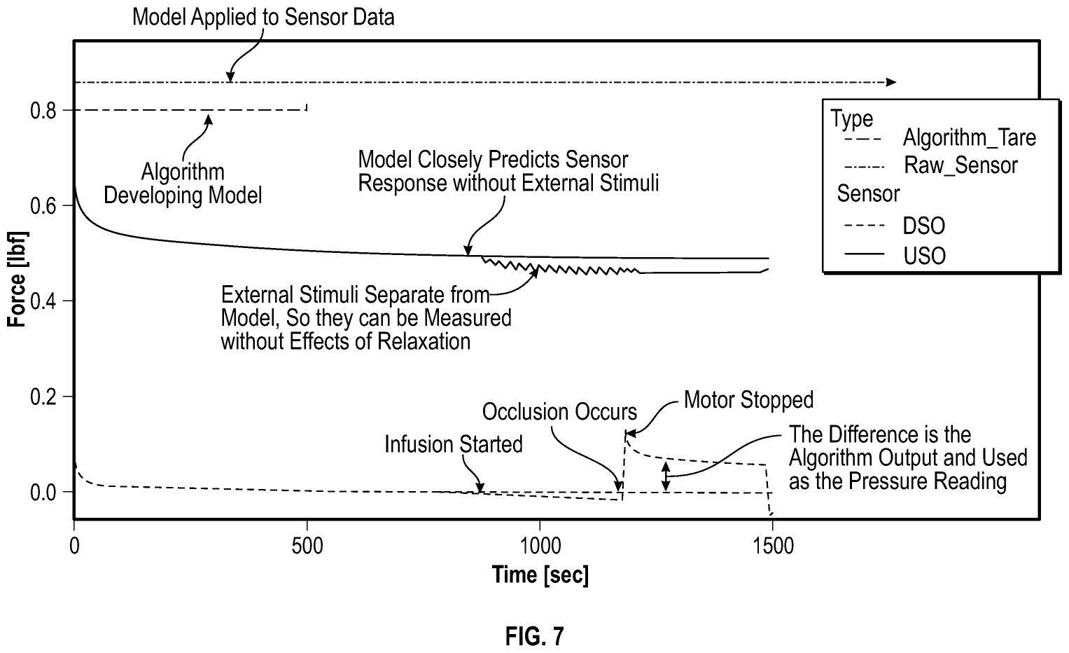

FIG. 7 graphically depicts a calculation of constants C₀and C₁ through the curve fitting method of least squares. Between the time when the receptacle door 112 is latched (t=0) and a specified end time (t=500 seconds), the control unit 128 adjusts constants C₀, C₁, C₂, and C₃ until the stress relaxation function matches the observed data. Although an initial trial period of 500 seconds is utilized in this embodiment, other initial trial periods of time can be utilized. For example, in an embodiment, the trial period of time can be as short as five seconds.

After the specified end time, the control unit 128 applies the stress relaxation function to values measured by the one or more sensors 124/126 to remove the effect of stress relaxation. In some embodiments, the fitted constants C₀, C₁, C₂, and C₃ can be checked against predefined acceptance criteria during operation to ensure that the algorithm is performing satisfactorily. If the acceptance criteria are not met, a default stress relaxation curve can be utilized so that a baseline level of stress relaxation is accounted for.

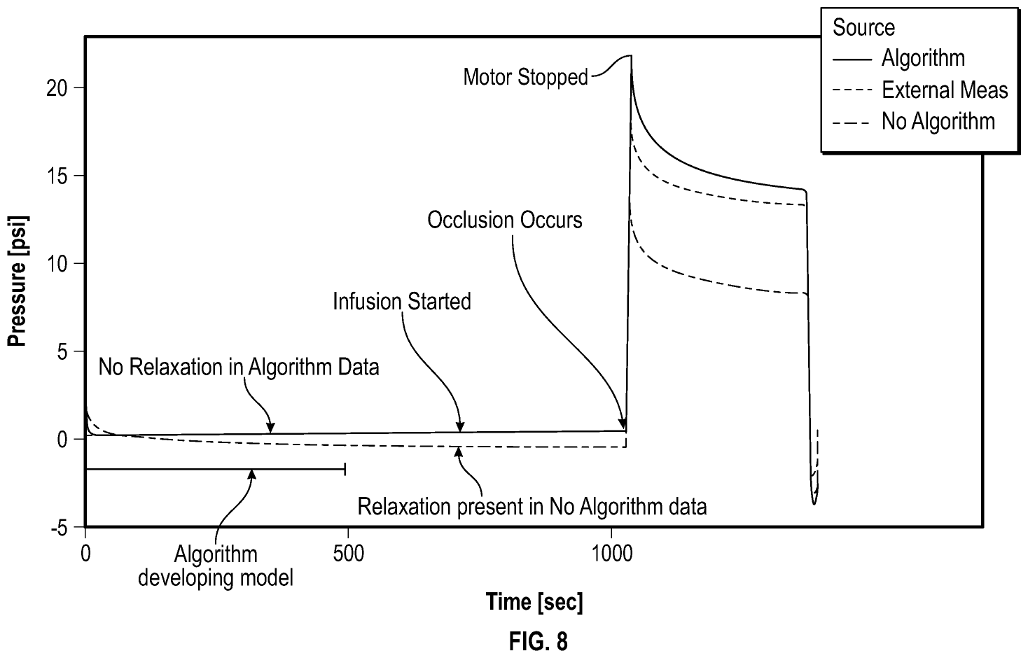

FIG. 8 depicts a graphical comparison between: a compensated pressure sensor value to account for stress relaxation (“[as stated on the graph]”); an uncompensated pressure sensor value (“[as stated on the graph]”); and data from an external source configured to directly monitor the infusate pressure (“[as stated on the graph]”). As depicted, the compensated pressure sensor closely corresponds to the actual fluid pressure of the infusate.

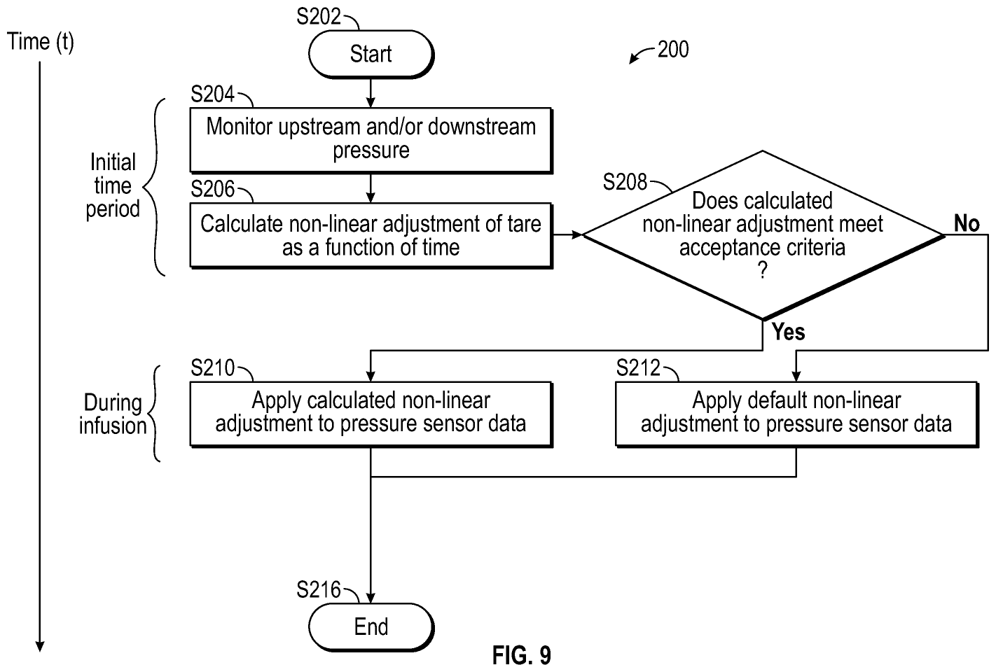

Referring to FIG. 9, a method 200 for compensating pressure measurement within an administration set 104 to account for stress relaxation effects is depicted in accordance with an embodiment of the disclosure. The method begins at S202. At S204, the system 100 monitors at least one of an upstream and downstream pressure via one or more sensors 124/126. At S206, the control unit 128 utilizes the data gathered by the one or more sensors 124/126 to calculate a nonlinear tare adjustment as a function of time as aforementioned. At S208, a determination is made whether the nonlinear tare adjustment calculated at S206 meets predefined acceptance criteria. If the calculated nonlinear tare adjustment meets predefined acceptance criteria, at S210 the calculated nonlinear tare adjustment is applied to the data gathered by the one or more sensors 124/126 to compensate for stress relaxation. If the calculated nonlinear tare adjustment does not meet the predefined acceptance criteria, at S212 a default nonlinear tare adjustment is applied to the data gathered by the one or more sensors 124/126. At S216, the process is complete.

Accordingly, embodiments of the present disclosure provide systems and methods to compensate for stress relaxation in the measurement of infusion pressure with respect to the tubing of an administration set, thereby enabling safer and more reliable measurement of infusate fluid pressures and an overall decrease in the amount of time necessary to detect an occlusion.

Various embodiments of systems, devices, and methods have been described herein. These embodiments are given only by way of example and are not intended to limit the scope of the claimed subject matter. It should be appreciated, moreover, that the various features of the embodiments that have been described may be combined in various ways to potentially produce additional embodiments. Moreover, while various materials, dimensions, shapes, configurations and locations, etc. have been described for use with disclosed embodiments, others besides those disclosed may be utilized without exceeding the scope of the claimed subject matter.

Persons of ordinary skill in the relevant arts will recognize that the subject matter hereof may comprise fewer features than illustrated in any individual embodiment described by example above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art, if not contrary to teachings of the subject matter hereof. Moreover, elements described with respect to an embodiment can be implemented in other embodiments even when not described in such embodiments unless otherwise noted.

Although a dependent claim may refer in the claims to a specific combination with one or more other claims, other embodiments could also include a combination of the dependent claim with the subject matter of each other dependent claim or a combination of one or more features with other dependent or independent claims. Such combinations are proposed herein unless it is stated that a specific combination is not intended or is contrary to the disclosure of the subject matter herein.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

What is claimed is:

1. An infusion pump, comprising:
an administration set configured to provide a fluidic pathway between a supply of infusate and an infusion set;
at least one pressure sensor configured to sense a pressure of infusate within the administration set; and
a control unit configured to monitor the sensed pressure, and apply a calculated tare adjustment to the monitored pressure to compensate for a decay of observable stress within the administration set as a result of stress relaxation.

2. The infusion pump of claim 1, further comprising a pump drive mechanism configured to urge infusate through the administration set by temporarily compressing a segment of the administration set.

3. The infusion pump of claim 2, wherein the pump drive mechanism comprises a peristaltic drive mechanism.

4. The infusion pump of claim 2, wherein the at least one pressure sensor comprises an upstream pressure sensor positioned upstream of the pump drive mechanism, a downstream pressure sensor positioned downstream of the pump drive mechanism, or a combination thereof.

5. The infusion pump of claim 1, wherein the control unit monitors the sensed pressure, in part, to detect the presence of an occlusion.

6. The infusion pump of claim 1, wherein the tare adjustment is calculated by the control unit based at least in part on data gathered by the at least one pressure sensor.

7. The infusion pump of claim 1, wherein the tare adjustment is represented by a nonlinear function.

8. The method of claim 1, wherein the tare adjustment is represented by a polynomial equation.

9. The method of claim 1, wherein the tare adjustment is represented by the equation: R(t)=C₀R(t)=C₀+C₁·t^−τ₁+C₂^(−t/τ₂)+C₃^(−t/τ₃), wherein R equals the stress relaxation as a function of time (t), τ₁, τ₂, τ₃, C₂ and C₃ represent hardware characterization constants, and C₀, and C₁ represent fitted constants.

10. The infusion pump of claim 1, wherein the tare adjustment is compared to acceptance criteria prior to application by the control unit.

11. An infusion system, comprising:
an infusion pump comprising drive mechanism, at least one pressure sensor and a control unit; and
an administration set configured to provide a fluidic pathway between a supply of infusate and an infusion set;
wherein the infusion pump is configured to monitor a pressure within the administration set as sensed by the at least one pressure sensor, and apply a calculated tare adjustment to the monitored pressure to compensate for a decay of observable stress within the administration set as a result of stress relaxation.

12. The infusion system of claim 11, wherein the infusion pump comprises at least one of a peristaltic pump or large volume pump.

13. The infusion system of claim 11, wherein the at least one pressure sensor comprises an upstream pressure sensor positioned upstream of the drive mechanism, a downstream pressure sensor positioned downstream of the drive mechanism, or a combination thereof.

14. The infusion system of claim 11, wherein the tare adjustment is represented by a nonlinear function.

15. A method, comprising:
monitoring at least one of an upstream pressure and downstream pressure of an administration set via one or more sensors;
calculating a tare adjustment based at least in part on data gathered from the one or more sensors;
determining whether the calculated tare adjustment meets acceptance criteria; and
applying the calculated tare adjustment to data gathered from the one or more sensors to compensate for a decay of observable stress within the administration set as a result of stress relaxation.
